(19) United States
(12) Patent Application Publication
(10) Pub. No.: US 2019/0269474 A1
(43) Pub. Date: Sep. 5, 2019

(54) MAGNET GUIDED WIRE CROSSING SYSTEM

(71) Applicant: Cook Medical Technologies LLC, Bloomington, IN (US)

(72) Inventors: Shaun Davis Gittard, Winston-Salem, NC (US); John Crowder Sigmon, JR., Winston-Salem, NC (US)

(21) Appl. No.: 16/272,020

(22) Filed: Feb. 11, 2019

Related U.S. Application Data
(60) Provisional application No. 62/636,884, filed on Mar. 1, 2018.

(51) Int. Cl. A61B 34/00; A61B 18/14

(52) U.S. Cl. CPC A61B 34/73; A61M 25/0127; A61B 2034/731; A61B 18/1492

(57) ABSTRACT

A magnet guided crossing system includes a first magnet that defines a first guide passage and a second magnet that defines a second guide passage, which are positioned in respective body structures. A crossing guide includes a catheter, a direction change piece and a magnetic latch in series that together define a third guide passage that extends from a proximal end of the catheter through a latching side of the magnetic latch. The poles of the first and second magnets and the magnetic latch are oriented so that the first and second magnets and the magnetic latch have a stable magnetically latched stack configuration. A puncture wire is slidably received in the catheter.

### GOVERNMENT RIGHTS

This invention was created in the performance of a Cooperative Research and Development Agreement with the National Institutes of Health, and Agency of the Department of Health and Human Services. The Government of the United States has certain rights in this invention.

### TECHNICAL FIELD

The present disclosure relates generally to crossing from one body structure to a second body structure with a wire, and more particularly to a magnet guided crossing system that includes at least two magnets.

### BACKGROUND

There are occasions during medical procedures where physicians need to exit one body structure and enter into another body structure with a catheter or wire. Examples include creating an arterio venous shunt, such as for dialysis or repair of congenital defects, arterio shunts, such as for treatment of pulmonary hypertension, venous access for transcatheter aortic valve replacement, transjugular intrahepatic portosystemic shunting (TIPS), esophageal altresia repair, and left atrial appendage stroke prevention (e.g., lareat device). There are also other locations and applications where this kind of crossing access is necessary. Current processes for creating these access routes are often risky, technically challenging, and often unfeasible in some applications, such as where tissues are moving or there is a distance between the body structures and their associated tissues.

One current process involves passing a wire unassisted out of one vessel towards another vessel, and then snaring the wire once it has entered the second vessel. This process is significantly easier and safer in end-to-end crossing procedures, where the path of crossing is in a more-or-less direct path from one structure to the other, such as in esophageal altresia. In situations where the trajectory of crossing is non-linear between the structures, such as a perpendicular trajectory crossing, crossing can become significantly more challenging since there is necessarily a bend in the crossing trajectory relative to the centerline of the body structure. In some of these technically challenging situations, surgery can often be deemed more practical instead of transcatheter procedures.

The present disclosure is directed toward one or more of the problems set forth above.

### SUMMARY OF THE DISCLOSURE

In one aspect, a crossing guide includes a catheter that defines a crossing lumen. The crossing guide also includes a direction change piece and a magnet that defines a hole therethrough. The catheter, the direction change piece, and the magnet are attached in series to define a guide passage that extends from a proximal end of the catheter through an exposed side of the magnet. The guide passage includes the lumen, the direction change piece and the hole. A distal end of the catheter is affixed to the direction change piece.

In another aspect, a magnet guided crossing system includes a first magnet that defines a first guide passage therethrough, and a second magnet that defines a second guide passage therethrough. A crossing guide includes a catheter, a direction change piece and a magnetic latch in series that together define a third guide passage that extends from a proximal end of the catheter through a latching side of the magnetic latch. Poles of the first and second magnets and the magnetic latch are oriented so that the first and second magnets and the magnetic latch have a stable magnetically latched stack configuration. A puncture wire is slidably received in the catheter.

In still another aspect, a method of crossing from a first body structure to a second body structure includes sandwiching tissue between a first magnet located in the first body structure and a second magnet located in the second body structure. A magnetic latch of a crossing guide is magnetically latched to the second magnet. A distal end of a puncture wire is moved from the first body structure into the second body structure by moving the puncture wire sequentially through a catheter of the crossing guide, a direction change piece of the crossing guide, the magnetic latch and the tissue.

### BRIEF DESCRIPTION OF THE DRAWINGS

FIG. 1 is a side schematic view of a crossing guide according to one embodiment of the present disclosure;

FIG. 2. Is a sectioned schematic view of the crossing guide of FIG. 1 as viewed along section lines 2-2;

FIG. 3 is a sectioned schematic view of a magnet guided crossing system using the crossing guide of FIGS. 1 and 2;

FIG. 4 is a schematic side view of a magnet guided crossing system using a crossing guide according to another embodiment of the present disclosure;

FIG. 5 is a right-side schematic view of the magnet guided crossing system of FIG. 4;

FIG. 6 is a sectioned schematic view of the magnet guided crossing system of FIG. 4 after being shifted into its crossing configuration and showing an electrified crossing instrument according to the present disclosure;

FIG. 7 is a step in a procedure for setting up the magnet guiding crossing system of the previous figures;

FIG. 8 shows the first and second magnets of FIG. 7 after becoming latched with tissue pinched therebetween.

FIG. 9 is a view similar to FIG. 8 expect after the tissue between the magnets has become more compressed into a membrane;

FIG. 10 shows another step of the procedure where the crossing guide of the present disclosure is being brought into the vicinity of the first and second magnets;

FIG. 11 shows the magnet guided crossing system after the magnets have achieved a stable magnetically latched stack configuration.

### DETAILED DESCRIPTION

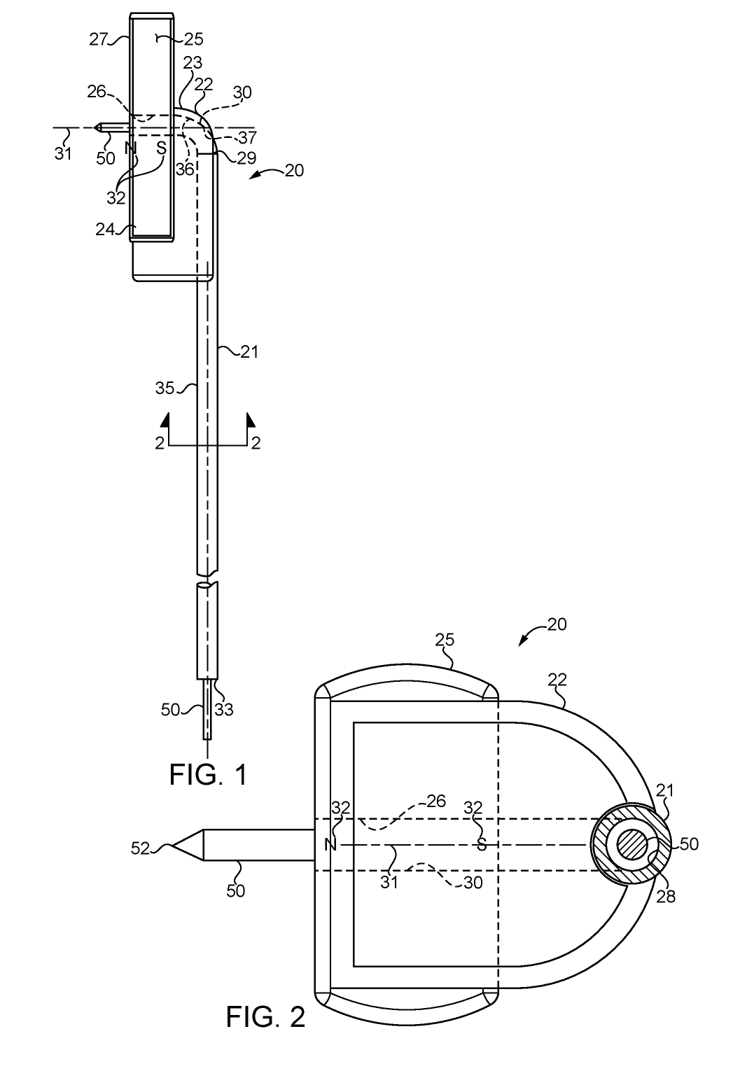

Referring initially to FIGS. 1 and 2, a crossing guide 20 according to the present disclosure includes a catheter 21 that defines a crossing lumen 28. The crossing guide 20 also includes a direction change piece 23 and a magnet 25 that defines a hole 26 therethrough. Catheter 21, direction change piece 23 and magnet 25 are attached in series to define a guide passage 30 that extends from a proximal end 33 of catheter 21 through an exposed side 27 of magnet 25. The guide passage 30 includes the crossing lumen 28, the direction change piece 23 and the hole 26. A distal end 29 of catheter 21 is affixed to the direction change piece 23. The direction change piece 23 and the distal end 29 of catheter 21 may be attached to a housing 22 that is sized to slide through a sheath. Preferably, housing 22 is sized to slide through a sheath with an inner diameter that is less than 7 millimeters. Magnet 25 has poles 32 that that are coincident with centerline 31. Catheter 21, direction change piece 23 and housing 22 may all be made from suitable medical grade plastics and attached to one another in a manner known in the art. In this embodiment, the magnet 25, the direction change piece 23 and the distal end 29 of catheter 21 may have fixed positions with respect to housing 22. In other words, these components are fixed to one another in a manner that does not permit relative movement. Direction change piece 23 may include an affixed ramp 36 that includes a guide surface 37 that turns through about a right angle. The term “about a right angle” means that when the angle is rounded off to an integer number of degrees that ends in zero, that the angle is 90°. For instance, 84° is not about a right angle, but 85° is about a right angle according to the present disclosure. The guide surface 37 may define a smooth curve with a radius that will guide a puncture wire to bend about a right angle without buckling.

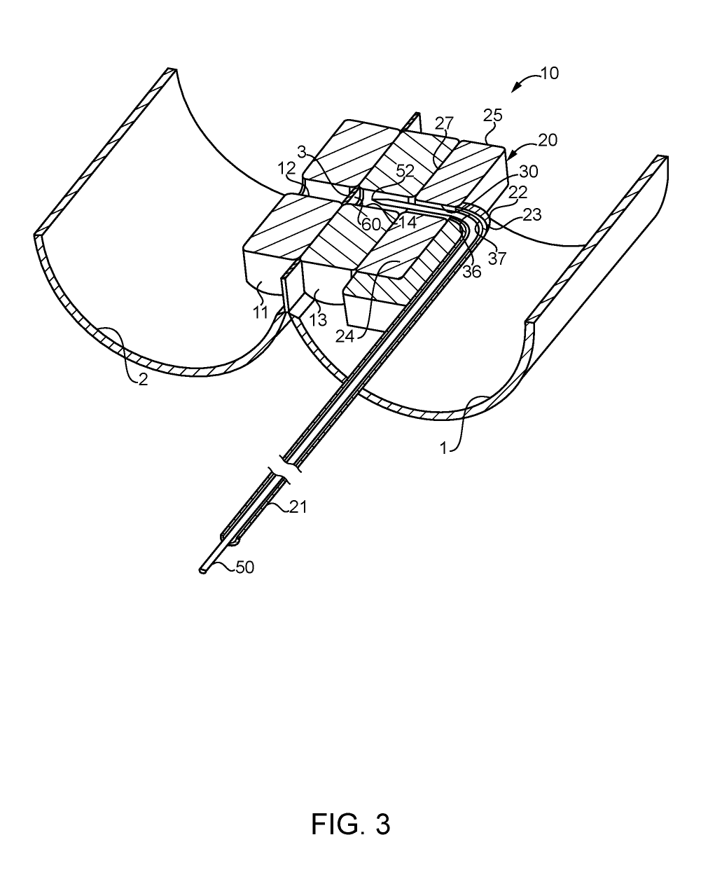

Referring now in addition to FIG. 3, when the crossing guide 20 of FIGS. 1 and 2 is magnetically joined with two other magnets 11, 13, the assembly may be considered a magnet guided crossing system 10, according to the present disclosure. FIG. 3 shows the magnet guided crossing system 10 being used to facilitate crossing a puncture wire 50, which is slidably received in catheter 21, from a first body structure 1 to a second body structure 2, which may typically be associated with passageways in the vicinity of, and/or chambers of, the heart. However, body structures 1 and 2 may be artificial for purposes of demonstration and teaching. Magnet guided crossing system 10 includes a first magnet 11 that defines a first guide passage 12 therethrough, and a second magnet 13 that defines a second guide passage 14 therethrough. The crossing guide 20, which includes catheter 21, direction change piece 23 and a magnetic latch 24 in series together to define a third guide passage 30 that extends from the proximal end 33 of catheter through a latching side 27 of magnetic latch 24. When magnetic latch 24 was first introduced in relation to crossing guide 20 of FIGS. 1 and 2, it was referred to as a magnet 25. Nevertheless, those skilled in the art will appreciate that magnetic latch 24 can also comprise a ferromagnetic block that is configured to stably latch to second magnet 13 in the configuration shown. In particular, the poles 40 of the first and second magnets 11, 13 and the magnetic latch 24, which may also be a third magnet 25, are oriented so that the first and second magnets 11, 13 and the magnetic latch 24 have a stable magnetically latched stack configuration 41 as shown in FIG. 3. FIG. 3 shows a membrane 60 sandwiched between first magnet 11 and second magnet 13 in the stable magnetically latched stack configuration 41. Membrane 60 may comprise tissue that defines portions of the respective body structures 1 and 2, or may comprise and artificial membrane when the magnet guided crossing system 10 is being used for demonstration or teaching purposes.

Although not necessary, a centerline 35 of the catheter 21 may be parallel to, but offset from the latching side 27 of third magnet 25 (magnetic latch 24). The embodiment of FIGS. 1-3 show a puncture wire 50 that includes a sharp piercing tip 52. In this embodiment, puncture wire 50 should have sufficient column stiffness when supported by catheter 21 to be able to pierce through membrane 60 when the puncture wire 50 is pushed and slid in a distal direction. Nevertheless, those skilled in the art will appreciate that any wire that includes structure that differentiates it from a conventional guide wire to facilitate puncturing through tissue could be considered a puncture wire in the context of the present disclosure. A puncture wire according to the present disclosure means something other than a wire guide. When the crossing guide 20 is configured for use with a sharp tipped puncture wire 50, as shown in FIGS. 1-3, the guide surface 37 of the direction change piece 23 may be puncture proof with respect to the puncture wire 50 such that the puncture wire 50 bends responsive to contact with the guide surface 37, including contact with the sharp piercing tip 52.

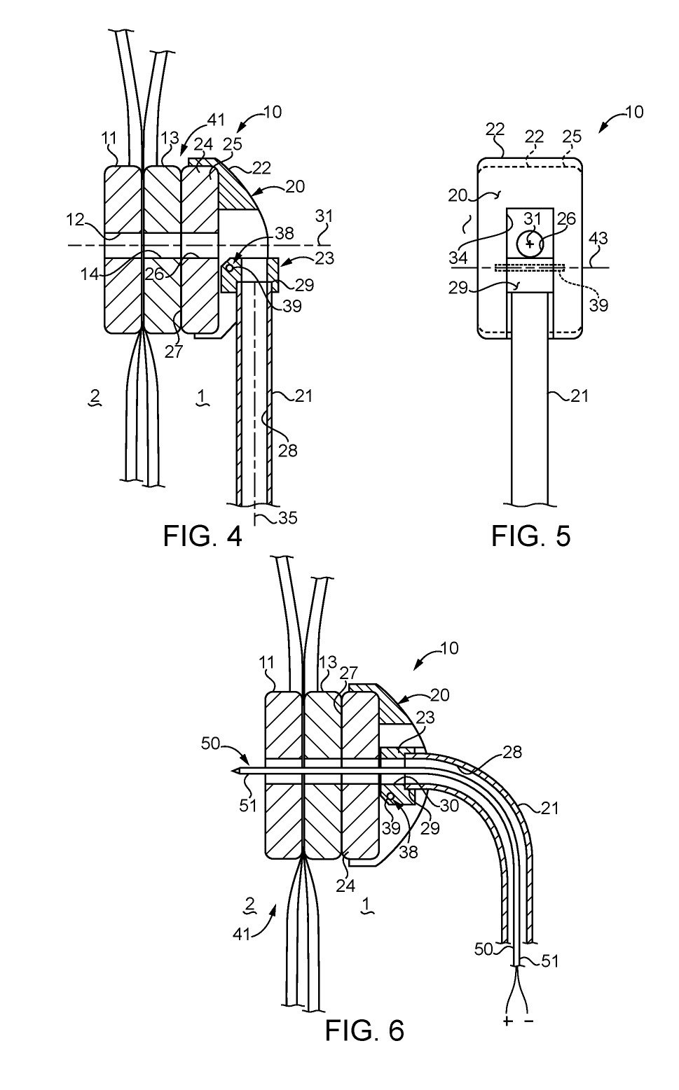

Referring now to FIGS. 4-6, a magnet guided crossing system 10 according to another embodiment of the present disclosure is illustrated using the same set of numerals to identify identically named features as in the previous embodiment. The magnet guided crossing system 10 includes first magnet 11 that defines a first guide passage 12 therethrough, and a second magnet 13 that defines a second guide passage 14 therethrough. FIGS. 4 and 6 show the first and second magnets 11, 13 sandwiching tissue that separates a first body structure 1 one from a second body structure 2, which may be real or artificial for purposes of demonstration or teaching. Like the earlier embodiment, the magnet guided crossing system 10 includes a crossing guide 20 that includes a catheter 21, a direction change piece 23 and a magnetic latch 24, which may be a third magnet 25, in series that together define a third guide passage 30 (FIG. 6) that extends from a proximal end (not shown) of catheter 21 through a latching side 27 of the magnetic latch 24. Like the earlier embodiment, the poles of the first and second magnets 11, 13 and the third magnet 25 (magnetic latch 24) are oriented so that the first and second magnets and the third magnet 25 have a stable magnetically latched stack configuration 41 as best shown in FIGS. 4 and 6. A puncture wire 50, which is illustrated as an electrified crossing instrument 51, is slidably received in catheter 21. This embodiment differs from the earlier embodiment in that direction change piece 23 is hinge mounted in housing 22, rather than having a fixed position as in the earlier embodiment. In particular, direction change piece 23 includes a hinge 38 that is defined by a hinge pin 39 that extends across a slot 34 defined by housing 22, as shown in FIG. 5. As in the previous embodiment, the distal end 29 of catheter 21 is affixed to the direction change piece 23. FIG. 4 shows a low profile configuration suitable for sliding the crossing guide 20 through a sheath to facilitate latching with the first and second magnets 11-13. Thereafter, the sheath may be withdrawn and the catheter 21 pushed in compression toward third magnet 25 to cause the direction change piece to rotate about the hinge axis 43 defined by hinge pin 39 through about a right angle to the configuration shown in FIG. 6. Thus, the distal end 29 of catheter 21 is attached to rotate with the hinge 38 from the orientation shown in FIG. 4 to the orientation shown in FIG. 6. The hinge pin 39 may be oriented at about a right angle with respect to centerline 31 of hole 26 as best shown in FIGS. 4 and 5. In addition, the hinge pin 39 and its defined hinge axis 43 may be offset from a centerline 35 of the lumen 28, as best shown in FIG. 4. Like the earlier embodiment, the various components can be made from suitable medical grade plastics of a type known in the art, but hinge pin 39 may preferably be made from a metallic material without departing from the scope of the present disclosure.

### INDUSTRIAL APPLICABILITY

The present disclosure finds general applicability to any circumstance in which there is a desire or need to advance a wire through tissue separating two body structures. The present disclosure finds specific applicability to guided crossing between passageways and chambers in or around the vicinity of the heart.

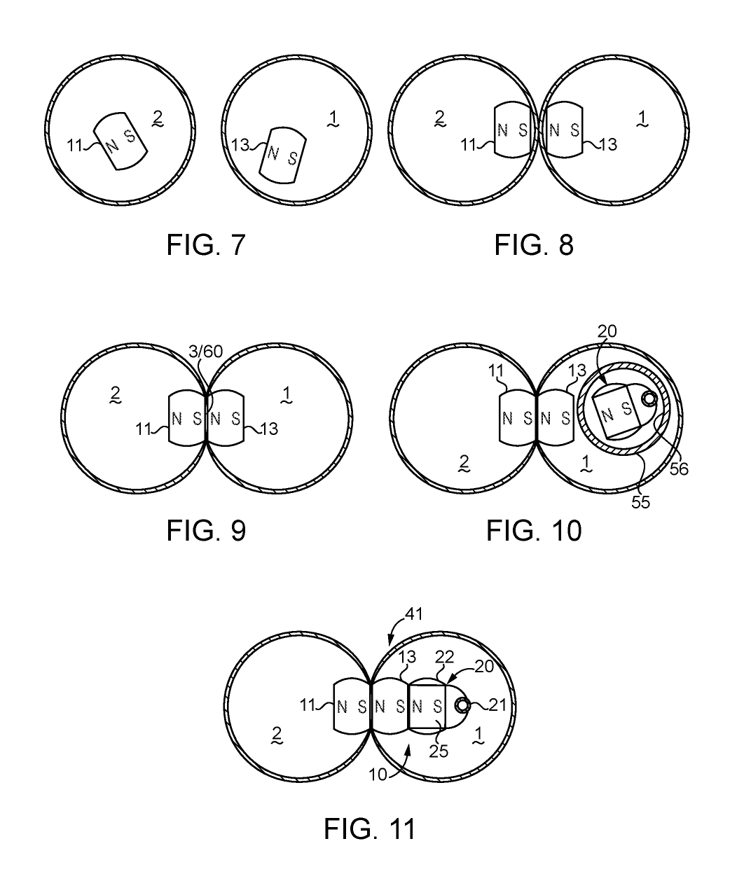

Referring now in addition to FIGS. 7-11, a series of schematics show a sequence of events leading to the magnet guided crossing system 10 achieving a stable magnetically latched stack configuration 41 to facilitate passage of a puncture wire from a first body cavity 1 to a second body cavity 2 according to the present disclosure. As shown in FIG. 7 the procedure may begin by positioning first and second magnets 11 and 13 in the respective body structures 1 and 2, and then guiding the magnets to latch to one another in a manner known in the art as per FIG. 8. Thereafter, the magnets tend to compress the tissue separating the magnets 11 and 13 to sandwich tissue 3 between the first magnet 11 and the second magnet 13. Those skilled in the art will appreciate that several prior art references teach magnets and strategies for achieving the configuration shown in FIG. 9, such as to necrose tissue separating the body structures 1 and 2 in order to create a passageway between the body structures. Thus, much of what occurs in FIGS. 7-9 is taught in the prior art and need not be taught again here.

After the first and second magnets 11 and 13 have been configured to sandwich tissue 3 between the first and second magnets 11 and 13, a sheath 55 may be positioned in the first body structure 1 in order to slide the crossing guide 20 of the present disclosure into position. Preferably, various components are sized such that the crossing guide 20 can be slid through a sheath 55 having an inner diameter that is less than 7 millimeters. Nevertheless, the present disclosure contemplates further miniaturization to utilize the invention in other areas of the body away from passageways and structures associated with the heart. Also, the present disclosure also contemplates larger structures that may be used, such as for instance in association with the gastrointestinal tract without departing from the present disclosure. After either withdrawing the sheath 55 in a proximal direction or by advancing the crossing guide 20 out the end of the sheath 55, the magnet 25 tends to snap into and find the stable latched configuration 41 as shown in FIG. 11. Thereafter, if using the embodiment shown in FIGS. 4-6, the catheter 21 may be advanced toward magnet 25 to create the configuration shown in FIG. 6. When ready, the puncture wire 50, which may have a sharp piercing tip 52, or comprise an electrified crossing instrument 51, or both, is slid through catheter 21 and then sequentially through the crossing guide 20, the direction change piece 23, the third magnet 25 and through the membrane 60 or tissue 3 separating the first and second magnets 11 and 13. Those skilled in the art will appreciate that the puncture wire 50 may bend at about a right angle responsive to moving the distal end of the puncture wire 50 from the first body structure 1 to the second body structure 2. As best illustrated in the sequence FIG. 7-11, the step of sandwiching the tissue between the first magnet 11 and the second magnet 13 is performed prior to magnetically latching the crossing guide 20 to the second magnet 13. If the embodiment of FIGS. 4-6 is being utilized, the direction change piece 23 will be pivoted about hinge axis 43 responsive to pushing a proximal end of catheter 21 toward the distal end 29 of the catheter.

The present description is for illustrative purposes only, and should not be construed to narrow the breadth of the present disclosure in any way. Thus, those skilled in the art will appreciate that various modification might be made to the presently disclosed embodiments without departing from the full and fair scope and spirit of the present disclosure. Other aspects, features and advantages will be apparent upon an examination of the attached drawings and appended claims.

What is claimed is:

1. A magnet guided crossing system comprising:
a first magnet that defines a first guide passage therethrough;
a second magnet that defines a second guide passage therethrough;
a crossing guide that includes a catheter, a direction change piece and a magnetic latch in series that together define a third guide passage that extends from a proximal end of the catheter through a latching side of the magnetic latch;
wherein poles of the first and second magnets and the magnetic latch are oriented so that the first and second magnets and the magnetic latch have a stable magnetically latched stack configuration; and
a puncture wire slidably received in the catheter.

2. The crossing system of claim 1 wherein the magnetic latch includes a third magnet.

3. The crossing system of claim 1 wherein a centerline of the catheter is parallel to, but offset from, the latching side of the magnetic latch.

4. The crossing system of claim 1 wherein the crossing guide is slidably received in a sheath having an inner diameter less than seven millimeters.

5. The crossing system of claim 1 including a membrane sandwiched between the first magnet and the second magnet in the stable magnetically latched stack configuration.

6. The crossing system of claim 1 wherein the puncture wire is an electrified crossing instrument.

7. The crossing system of claim 1 wherein the direction change piece includes an affixed ramp with a guide surface that defines a portion of the third guide passage; and
the guide surface is puncture proof with respect to the puncture wire such that the puncture wire bends responsive to contact with the guide surface.

8. The crossing system of claim 1 wherein the direction change piece includes a hinge mounted to rotate with respect to the magnetic latch; and
a distal end of the catheter is attached to rotate with the hinge.

9. A crossing guide comprising:
a catheter that defines a crossing lumen;
a direction change piece;
a magnet that defines a hole therethrough;
the catheter, the direction change piece and the magnet being attached in series to define a guide passage that extends from a proximal end of the catheter through an exposed side of the magnet, and the guide passage includes the lumen, the direction change piece and the hole; and
wherein a distal end of the catheter is affixed to the direction change piece.

10. The crossing guide of claim 9 wherein a centerline of the hole is parallel to a pole of the magnet.

11. The crossing guide of claim 10 wherein the magnet, the direction change piece and a distal end of the catheter are all attached to a housing that is sized to slide through a sheath with an inner diameter that is less than seven millimeters.

12. The crossing guide of claim 11 wherein magnet, the direction change piece and the distal end of the catheter have fixed positions with respect to the housing.

13. The crossing guide of claim 12 wherein the direction change piece includes a guide surface that turns through about a right angle.

14. The crossing guide of claim 12 wherein the distal end of the catheter is affixed to the direction change piece; and
the direction change piece is mounted to rotate about a hinge pin attached to the housing.

15. The crossing guide of claim 14 wherein the hinge pin is oriented at about a right angle with respect to a centerline of the hole; and
the hinge pin is offset from a centerline of the lumen.

16. A method of crossing from a first body structure to a second body structure comprising the steps of:
sandwiching tissue between a first magnet located in the first body structure and a second magnet located in the second body structure;
magnetically latching a magnetic latch of a crossing guide to the second magnet; and
moving a distal end of a puncture wire from the first body structure into the second body structure by moving the puncture wire sequentially through a catheter of the crossing guide, a direction change piece of the crossing guide, the magnetic latch and the tissue.

17. The method of claim 16 wherein the moving step includes moving the puncture wire through the second magnet and the first magnet.

18. The method of claim 16 including bending the puncture wire at about a right angle responsive to moving a distal end of a puncture wire from the first body structure into the second body structure.

19. The method of claim 16 wherein the sandwiching step is performed prior to the magnetically latching step.

20. The method of claim 16 including pivoting the direction change piece about a hinge axis responsive to pushing a proximal portion of the catheter toward a distal end of the catheter.
